(19) United States
(12) Patent Application Publication
(10) Pub. No.: US 2018/0132703 A1
(43) Pub. Date: May 17, 2018

(54) DISPOSABLE MEDICAL SYSTEMS, DEVICES, AND RELATED METHODS

(71) Applicant: Boston Scientific Scimed, Inc., Maple Grove, MN (US)

(72) Inventors: Kenneth REEVER, Hopedale, MA (US); Richard TAH, Milford, MA (US); Brian MacLEAN, Westford, MA (US); Thomas ZAPPIA, West Boylston, MA (US); Maria MAILLET, Hudson, MA (US); Zakh SOKOLOVSKIY, Brighton, MA (US); Peter BOROS, Ellettsville, IN (US); Joseph ARRUDA, Taunton, MA (US)

(73) Assignee: Boston Scientific Scimed, Inc., Maple Grove, MN (US)

(21) Appl. No.: 15/809,142

(22) Filed: Nov. 10, 2017

Related U.S. Application Data
(60) Provisional application No. 62/420,938, filed on Nov. 11, 2016.

(51) Int. Cl. A61B 1/00; A61B 1/05; A61B 1/06

(52) U.S. Cl. CPC A61B 1/00105; A61B 1/00066; A61B 1/00096; A61B 1/018; A61B 1/053; A61B 1/0676; A61B 1/00101; A61B 1/00128

(57) ABSTRACT

An insertion device may include a hollow handle and a shaft extending distally of the hollow handle. The insertion device may also include a cap coupled to a distal end of the shaft. The cap may include a camera and an LED coupled thereto.

### CROSS-REFERENCE TO RELATED APPLICATIONS

This patent application claims the benefit of priority under 35 U.S.C. § 119 to U.S. Provisional Patent Application No. 62/420,938, filed Nov. 11, 2016, which is herein incorporated by reference in its entirety.

### TECHNICAL FIELD

Various aspects of the present disclosure relate generally to medical systems, devices, and related methods. More specifically, the present disclosure relates to disposable medical systems and/or devices.

### BACKGROUND

The field of endoscopy covers systems and methods a user may employ to examine and/or treat a subject with, e.g., the assistance of an endoscope or other suitable introduction sheaths or devices. An endoscope (or other suitable introduction device) may provide for viewing of, for example, the interior of a hollow organ or cavity in the subject's body (e.g., the gastrointestinal (GI) tract, the urinary tract, the respiratory tract, etc.). Ureteroscopy, for example, is a subset of endoscopy. Ureteroscopy may include procedures in which an endoscope (such as, e.g., a ureteroscope, uteroscope, cystoscope, hysteroscope, etc.) may be passed through the subject's urethra and bladder, and directly into the subject's ureter. The endoscope may be further inserted into the subject's kidney for pyeloscopic procedures. These procedures may be useful in the diagnosis and the treatment of disorders of the subject's urinary tract, such as the presence of kidney stones that may block urinary tract ducts.

Sterilization of such endoscopes is imperative to avoid cross-contamination between patients. Unfortunately, insufficient sterilization of endoscopes is a persistent, and hazardous problem in the medical device industry. Not only is it difficult for hospitals and medical professionals to reprocess or sterilize a wide array of endoscopes effectively, it's also costly. Moreover, typical endoscopes are expensive to manufacture, requiring highly precise and often individual endoscope-specific components (e.g., camera, illumination features, various medical tool adaptors, etc.) resulting in devices exceeding tens of thousands, and possibly hundreds of thousands of dollars. Thus, such devices are impractical for use as a one-time-use disposable device.

The systems, devices, and methods of the current disclosure may rectify some of the deficiencies described above or address other aspects of the prior art.

### SUMMARY

Examples of the present disclosure relate to, among other things, medical devices. Each of the examples disclosed herein may include one or more of the features described in connection with any of the other disclosed examples.

In one example, an insertion device may include a hollow handle and a shaft extending distally of the hollow handle. The insertion device may also include a cap coupled to a distal end of the shaft. The cap may include a camera and an LED coupled thereto.

Examples of the insertion device may include one or more of the following features. The shaft may be a rigid shaft. The shaft may include a multi-lumen extrusion. The shaft may include a plurality of discrete lumens fixedly coupled together. The hollow handle may include a rounded ball-head and an extension depending therefrom. The shaft may extend from a location within the ball-head and through a side of the ball-head of the hollow handle. A port may extend from a side of the ball-head of the hollow handle opposite the side from which the shaft extends. The hollow handle may include a plug extending from a portion of the extension of the hollow handle opposite the ball-head. The plug may be selectively coupleable and uncouplable from an umbilicus connector. The handle may include two fluid ports proximate the plug. Each of the two fluid ports may be fluidly coupled to a tube extending through the hollow handle, through the shaft, and toward the cap. The umbilicus may be coupled to a computer. The LED may be a first LED, the insertion device may further include a second LED disposed on the cap. Each of the first and second LEDs may be disposed on opposing sides of the camera. A working channel may extend through and proximally of a proximal end of the shaft.

In a further example, an insertion device may consist essentially of a handle including a head having a port which may extend from a first side of the head and a grip which may depend from the head. The insertion device also may include a shaft extending distally of the hollow handle and a cap coupled to a distal end of the shaft. The cap may include a camera and a light sourced coupled thereto.

Examples of the insertion device may further include one or more of the following features. A proximal end of the shaft may be disposed within the rounded ball-head of the handle. A plug may extend from the grip in a direction opposite of the ball-head, and the plug may be selectively coupleable and uncouplable from an umbilicus connector. The grip may include two ports proximate the plug, and each of the two ports may be fluidly coupled to a tube extending through the handle, through the shaft, and toward the cap.

In a further example, an insertion device may include a handle. The handle may consist essentially of a rounded ball-head having a port extending from a first side of the rounded ball-head and a grip depending from the rounded ball-head, the grip including a pair of fluid ports and a plug extending from the grip in a direction opposite that of the rounded ball-head. The insertion device may further include a shaft having a proximal end disposed within the rounded ball-head and extending from a second side of the ball-head in a direction opposite the first side of the rounded ball-head. A cap may be coupled to a distal end of the shaft and the cap may include a camera and a light source coupled thereto.

Examples of the insertion device may include one or more of the following features. A proximal end of a working channel may be coupled to the port and may extend distally through a proximal end of the shaft and towards the cap. The handle may include a plug extending from a portion of the extension of the handle opposite the ball-head, and wherein a cable or a bundle of cables may have a first end coupled to at least one of the camera and light source and a second end coupled to the plug.

Both the foregoing general description and the following detailed description are exemplary and explanatory only and are not restrictive of the features, as claimed. As used herein, the terms “comprises,” “comprising,” or other variations thereof, are intended to cover a non-exclusive inclusion such that a process, method, article, or apparatus that comprises a list of elements does not include only those elements, but may include other elements not expressly listed or inherent to such a process, method, article, or apparatus. Additionally, the term “exemplary” is used herein in the sense of “example,” rather than “ideal.” As used herein, the terms “about,” “substantially,” and “approximately,” indicate a range of values within +/−5% of a stated value.

### BRIEF DESCRIPTION OF THE DRAWINGS

The accompanying drawings, which are incorporated in and constitute a part of this specification, illustrate exemplary features of the present disclosure and together with the description, serve to explain the principles of the disclosure.

FIG. 1 illustrates an exemplary disposable insertion device according to aspects of the disclosure;

FIG. 2 illustrates a partial isometric view of a cap of the exemplary insertion device of FIG. 1;

FIG. 3 illustrates a partial cross-sectional view of the insertion device of FIG. 1;

FIG. 4 illustrates a cross-sectional view of a shaft of the insertion device of FIG. 1, according to aspects of the disclosure; and

FIG. 5 illustrates a cross-sectional view of a shaft of an insertion device, according to additional aspects of the disclosure.

### DETAILED DESCRIPTION

Examples of the present disclosure relate to medical systems and devices for diagnosing and/or treating internal areas of a subject's body. The medical system may include an insertion device and one or more medical devices operably coupled thereto for introduction of an end-effector or other object through the insertion device.

Reference will now be made in detail to examples of the present disclosure described above and illustrated in the accompanying drawings. Wherever possible, the same reference numbers will be used throughout the drawings to refer to the same or like parts.

The terms “proximal” and “distal” are used herein to refer to the relative positions of the components of an exemplary medical device or insertion device. When used herein, “proximal” refers to a position relatively closer to the exterior of the body or closer to an operator using the medical device or insertion device. In contrast, “distal” refers to a position relatively further away from the operator using the medical device or insertion device, or closer to the interior of the body.

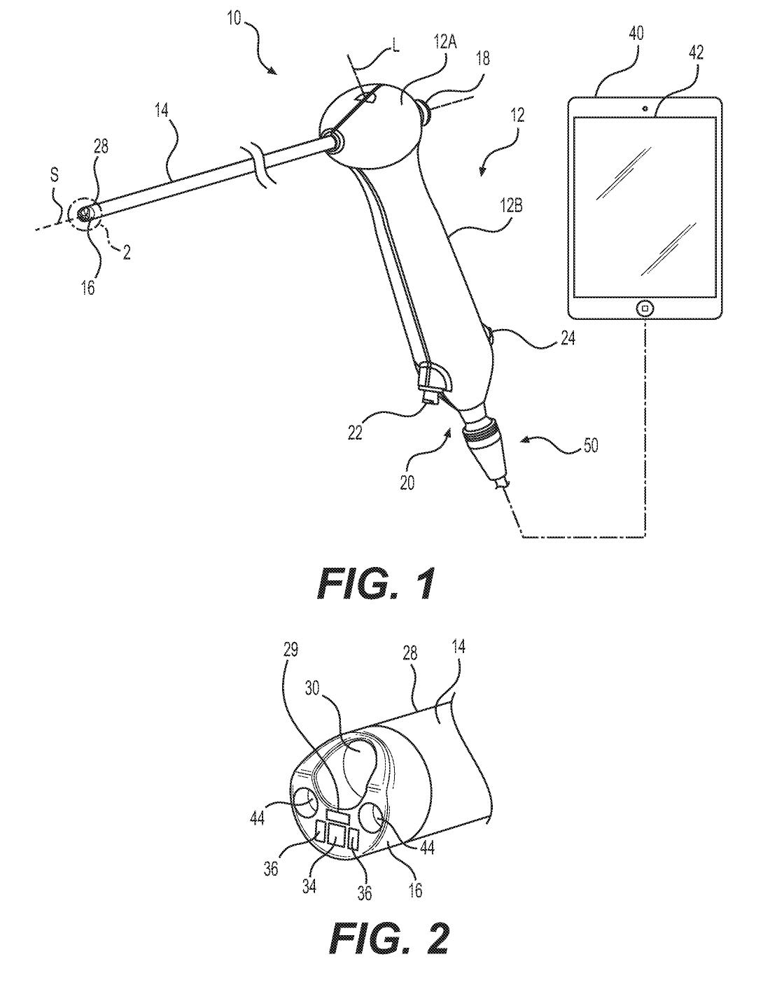

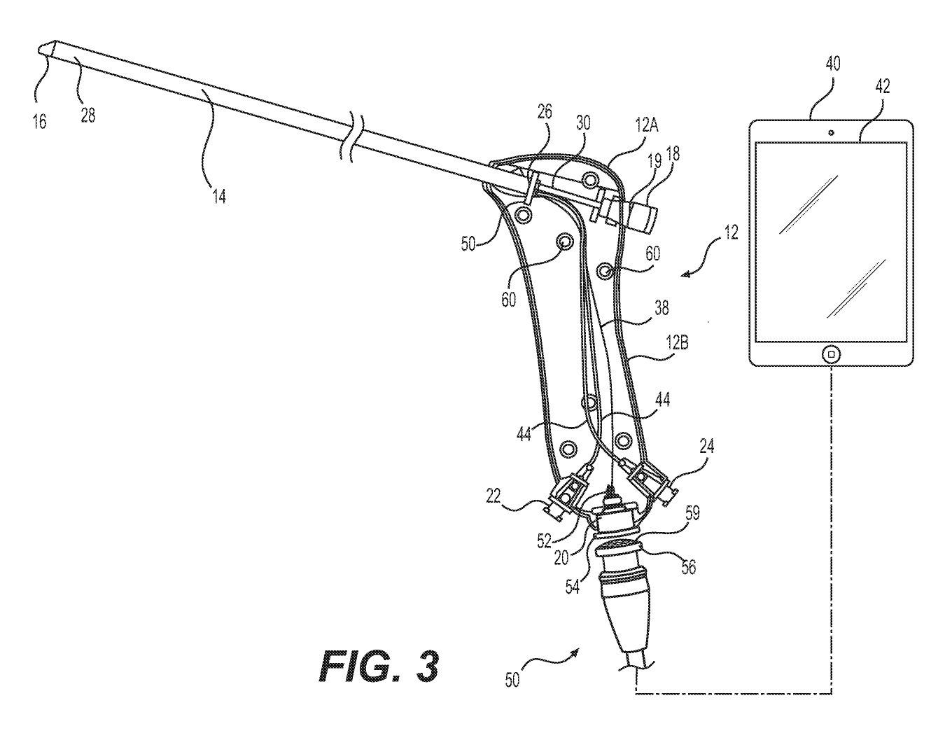

FIGS. 1 and 2 illustrate an exemplary insertion device 10 including a hollow handle 12 coupled to a shaft 14 terminating in a cap 16. Handle 12, as will be described in further detail below, is a hollow or empty handle in which only shaft 14, first and second tubes 44, a working channel 30, a cable/bundle 38, ports 18, 22, and 24, and a plug 20 extends or resides therein. Additionally, one or more molding remnants, flashings, or handle connection members 60 (FIG. 3) for connecting portions of the handle together may be present within hollow handle 12. For example, connection members 60 may be used to hold the two handle halves together after assembly. They may contain screws, function as adhesive bonding pads, ultrasonic weld points, friction fit points, and/or snap fits. Alternatively, connection members 60 may be absent from insertion device 10. In such a manner, hollow handle 12 is devoid of any additional functional components (e.g., heat sinks, circuit boards, illumination sources, steering cables or actuators, processing equipment, and/or additional circuitry) found in prior art devices. As such, hollow handle 12 is relatively inexpensive, light, and uncluttered or unencumbered as compared to prior art handles. In other words, hollow handle 12 refers to a device containing only working channel 30, first and second tubes 44, cable/bundle 38, plug 20, and ports 18, 22, and 24, and does not include heat sinks, circuit boards, illumination sources, steering cables or actuators, processing equipment or additional circuitry, as will be described in further detail below. That is, handle 12 may consist essentially of shaft 14, first and second tubes 44, working channel 30, a cable/bundle 38, ports 18, 22, and 24, and a plug 20 while not including any additional hardware components such as, e.g., heat sinks, circuit boards, illumination sources, steering cables or actuators, processing equipment, and/or additional circuitry. Additionally, while hollow handle 12 is generally devoid of a light source housed therein, in an alternative embodiment, a partially hollow handle 12 may include one or more LEDs 36 therein, and include one or more optical fibers extending along shaft 14 for conveying light distally of cap 16.

Cap 16 includes a rounded circumferential edge and is angled so as to be atraumatic to tissue. Additionally, cap 16 may be coupled (e.g., removably or permanently) to shaft 14 in any appropriate manner such as, for example, snap fit, interference fit, adhesives, welding, molding, etc. Insertion device 10 includes any device configured to allow an operator to perform medical diagnoses and/or treatments on a subject. For example, insertion device 10 includes any device configured to allow a user to access and view internal areas of a subject's body. For example, insertion device 10 may be inserted into any portion of a urinary tract, such as a ureter, a gastrointestinal lumen, such as an esophagus; a vascular lumen; and/or an airway.

According to aspects of the present disclosure, insertion device 10 is a ureteroscope. In some contemplated examples, insertion device 10 is a sterile, single-use, and completely disposable ureteroscope. That is, upon completion of a medical procedure, insertion device 10, including hollow handle 12, shaft 14, and cap 16 may be disconnected from any appropriate console or power source (e.g., computer 40) and thrown away. As such, concerns of cross-contamination or insufficient sterilization of insertion device 10 may be alleviated. Other types of devices, however, may be substituted for the ureteroscope, including, as examples, a hysteroscope, a uteroscope, a bronchoscope, a cystoscope, and similar devices. Any such devices may be single-use and completely disposable.

Hollow handle 12 of insertion device 10 may have any shape suitable for gripping and controlling insertion device 10. For example, hollow handle 12 may have an ergonomic shape designed to be held comfortably in the hand, e.g., the palm of the hand of a medical professional. For example, as shown in FIG. 1, hollow handle 12 includes an enlarged spherical, or substantially spherical ball-head 12A coupled to shaft 14 and a pistol grip or extension 12B depending therefrom. As shown, ball-head 12A includes a single (i.e., only one) port 18 on a proximal side thereof (e.g., a side of hollow handle 12 closest to a body of the medical professional when being held by the medical professional, and/or a side of ball-head 12A opposite the side from which shaft 14 extends), as will be described in further detail below. Additionally, a selectively coupleable and uncouplable plug 20 and ports 22 and 24 positioned on extension 12B are located on a proximal end thereof, e.g., on an end of extension 12B opposite the position of the ball-head 12A, as will be described in further detail below. In such a manner, a medical professional may grip hollow handle 12 along either the enlarged ball-head 12A or the extension 12B, depending on his/her preference. For example, in some arrangements, the medical professional may grip or hold hollow handle 12 by placing or wrapping the palm of his or her hand on or around a top surface (e.g., a surface of ball-head 12A opposite the extension 12B) of ball-head 12A, thereby avoiding interference with port 18 of ball-head 12A. Additionally or alternatively, in some arrangements, the medical professional may grip or hold hollow handle 12 by placing or wrapping the palm of his or her hand on or around the extension between the ball-head 12A and plug 20 and ports 22 and 24, such that interference with plug 20 and ports 18, 22, and 24, may be avoided.

As shown in FIG. 1, hollow handle 12 is bilaterally symmetric about a plane extending along the longitudinal axis L of hollow handle 12 and may include a clam shell arrangement. Shaft 14 may extend along a longitudinal shaft axis S from a proximal end 26 within hollow handle 12 (FIG. 3) to a distal end 28 coupled to cap 16. As shown in FIG. 3, proximal end 26 of shaft 14 is fixedly secured via attachment mechanism 50 (e.g., screws, bolts, or the like) to hollow handle 12 such that shaft is fixedly coupled (e.g., rotationally and axially immovably attached) to hollow handle 12. Shaft 14 is a rigid (e.g., non-bendable, deflectable, etc.) shaft. For example, shaft 14 may be comprises of one or more of the following materials: stainless steel, titanium, rigid plastic, glass fiber reinforced polymer resin (fiberglass), and hard coat anodized aluminum. Additionally, shaft 14 has a length extending from proximal end 26 to distal end 28 of between about 230 mm to about 335 mm.

As noted above, hollow handle 12 includes a plurality of ports. For example, port 18 extends from ball-head 12A parallel or aligned with longitudinal axis S and communicates with a working channel 30 extending through shaft 14 and tip 16, as shown in FIGS. 2 and 3. That is, working channel 30 extends from port 18 through shaft 14 and terminates in cap 16. Working channel 30 extends along an axis offset and parallel with longitudinal axis S and includes a radial size (e.g., diameter) smaller than a radial size (e.g., diameter) of shaft 14. Additionally, working channel 30 includes a radial size (e.g., diameter) larger than a radial size (e.g., diameter) of any other channel (e.g., first and second tubes 44, as will be described in further detail below) of insertion device 10. Additionally, although working channel 30 is illustrated and described as having a circular cross-sectional shape, the disclosure is not so limited. Rather, working channel 30 may have an oval, semi-circular, or any other cross-sectional shape that defines an inner passage way therethrough. Port 18 may facilitate the insertion of one or more medical device(s) through working channel 30 to distal end 28 of shaft 14 and cap 16. For example, port 18 may comprise a medical (e.g., threaded) or slip luer port through which an end-effector, control member, and/or sheath of a medical device (not shown) may be delivered into and/or through working channel 30. Port 18 may further include a one-way valve 19 (FIG. 3) which allows the passage (insertion and removal) of a medical device therethrough but prevents or limits the outlet of fluid from working channel 30.

As shown in FIG. 2, insertion device 10 includes an imaging assembly including a digital camera 34 at a distal end of cap 16. For example, camera 34 may be at a distalmost tip of cap 16 and/or may be at least partially embedded within cap 16. Camera 34 may view an area distal to the distal end 28 of shaft 14 and cap 16. Camera 34 may have any appropriate resolution to capture images and/or full-motion video images in digital or any other suitable format. For example, camera 34 may include a pixel count greater than about 20,000 pixels and less than about 80,000 pixels. For example, camera 34 may have a pixel count of about 62,500. Camera 34 may include a field of view of at least 80°. Additionally, camera 34 may include all those components (e.g., sensors, filters, signal converters) necessary for receiving and transmitting an image signal to a computer 40 via a cable 38, as will be described in further detail below. That is, camera 34 may contain a lens and a digital sensor to transmit a signal via cable 38 to computer 40 for processing and display.

Additionally, insertion device 10 includes an illumination unit including one or more (e.g., two) light-emitting diodes (“LED”) 36. LEDs 36 may be at a distalmost tip of cap 16 and/or may be at least partially embedded within cap 16. As shown, LEDs 36 may be located on opposite sides of camera 34 and may emit light upon receipt of an appropriate power supply. LED 36 may include, for example, a high-power sub 2 mm LED with a CCT range of 5000-6500K. Any other suitable LED may be used. Further, insertion device 10 includes a pressure sensor 29 at least partially embedded within cap 16. Pressure sensor 29 may be arranged to directly measure body cavity pressure of a patient. Pressure sensor 29 may include a piezoresistive pressure die (such as, e.g., the P330 Series 1F Absolute Pressure Sensor Die from Nova Sensor).

A bundle of cables 38 (or alternatively, a single cable 38 adapted for conveyance of both power and data) may be coupled to one or more of camera 34, LEDs 36, and pressure sensor 29 and extend proximally along shaft 14 between an outer wall of working channel 30 and an inner wall of shaft 14, and connect to plug 20. Plug 20 includes an electrical connector to connect hollow handle 12, including cable/bundle 38, to an umbilicus 50 for facilitating electrical connections and functions, such as transferring data to/from camera 34, pressure sensor 29, and/or powering LEDs 36. As shown in FIG. 3, for example, plug 20 may include one or more terminals 52 fixedly coupled to (e.g., soldered to) cable/bundle 38. Additionally, plug 20 may include an adaptor 54 for selectively coupling and uncoupling from an adaptor 56 of umbilicus 50.

To facilitate secure connection or mating between adaptors 54 and 56, adaptors 54 and 56 may be magnetically attracted toward one another, or threadably connected to maintain contact therebetween, or use a method of interfering parts (snap or lever release connection) to hold the mating halves together. Additionally or alternatively, at least one of adaptors 54 and 56 may include one or more leads (not shown) arranged to mate or otherwise connect with one or more receptacles 59 on the other of adaptors 54 and 56 so as to convey signals (e.g., power and/or data) from/to cable/bundle 38 to or from umbilicus 50, which is in turn coupled to a computer 40.

Computer 40 may include a smartphone, tablet computer, laptop computer, desktop computer, and/or any other suitable computing device. Computer 40 may include a display 42, as well as other electronic components (not shown), such as a central processing unit, memory, video and graphics cards, wireless and wired networking devices, audio devices, one or more input/output ports, a power supply, and/or any other suitable computer features. Display 42 may include a touch screen for displaying image data, and for receiving inputs or commands from a user. User interaction may be directed toward aspects of image capture, video capture, brightness control, mode controls, narrow band imaging toggle, and/or any other controls that may be part of a typical ureteroscopy procedure. Computer 40 may include imaging electronics configured to process and/or transfer the image data, display the image data on display 42 for viewing by a user, and send signals to camera 34 and LEDs 36.

Each of ports 22 and 24 may include a luer (or other mating configuration) fluidly coupled with one of first and second channels, passages, or tubes 44 extending through shaft 14 and tip 16, as shown in FIGS. 2 and 3. That is, each tube 44 extends from one of ports 22 and 24, through shaft 14, and terminates in cap 16. Any one or both of tubes 44 may be fluidly coupled with a source of irrigation fluid, a source of aspiration fluid (e.g., negative pressure), a source of medicament for infusion, etc. Additionally, in some arrangements one or both of tubes 44 may receive pressure sensor 29, therein. For example, in one arrangement, a first tube 44 is fluidly coupled with a source of irrigation fluid (not shown) while a second tube 44 is fluidly coupled with a source of aspiration fluid (not shown). In another arrangement, at least the first tube 44 is fluidly coupled with a source of irrigation fluid (not shown) while a second tube 44 is fluidly coupled with a source of medicament. While in some exemplary arrangements, only two tubes 44 are employed, in further arrangements, more or less tubes 44 may be used. For example, in some arrangements, a single (e.g., only one) tube 44 may be employed, while in still further arrangements, three or more tubes 44 may be used.

In any such arrangement, ports 22 and 24 are located on a proximal end thereof of extension 12B opposite the position of the ball-head 12A. As shown in FIGS. 1 and 3, ports 22 and 24 may be circumferentially spaced 180° apart about the longitudinal axis L of hollow handle 12. In other arrangements, however, ports 22 and 24 may be spaced apart at any appropriate circumferential angle, such as, between about 20° and about 340° arranged about longitudinal axis L of hollow handle 12.

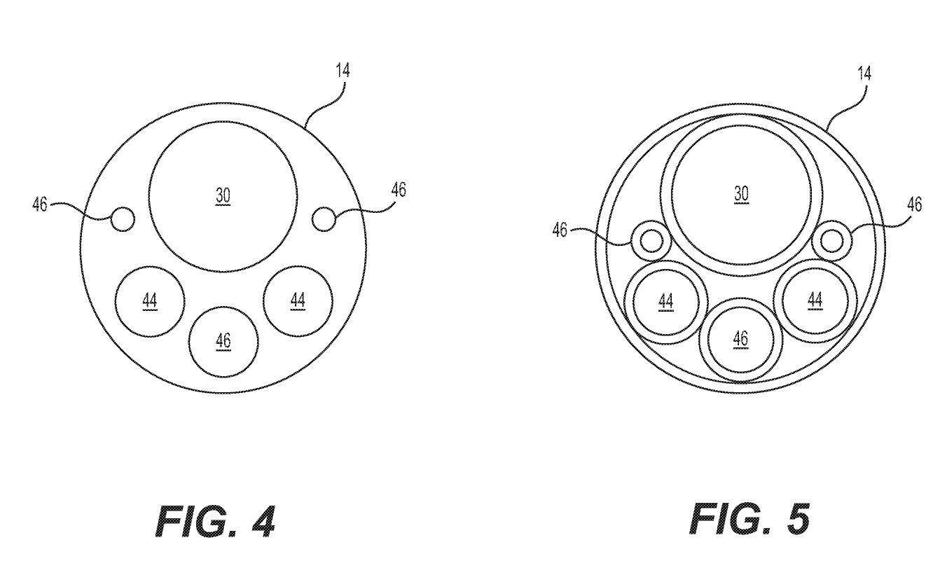

Shaft 14 of FIGS. 1-3 may be arranged as either a multi-lumen extrusion (FIG. 4) or as individual and discrete lumen or tubes secured together via, e.g., adhesives, welding, or the like (FIG. 5). In the arrangement shown in FIG. 5, spaced between adjacent lumens may optionally be filled with holding substance such as, for example, epoxy, adhesives, or the like. As shown in each of FIGS. 4 and 5, shaft 14 includes working channel 30 and first and second tubes 44. Optionally, shaft 14 may further include any or one or more cable lumens 46 (e.g., three cable lumens 46 are shown). In the case of the partial hollow handle in which LEDs 36 are located within handle 12, one or more of the cable lumens 46 may house or otherwise receive an optical fiber or the like for conveying light distally of shaft 14. Also, while each of lumens 30, 44, and 46 are illustrated as having a circular cross-section, the disclosure is not so limited. Rather, any one or more of lumens 30, 44, or 46 may have any appropriate shape including, for example, square, ovular, polygonal, and irregular shapes, or combinations thereof.

In use, a medical professional may grasp hollow handle 12 (e.g., either via ball-head 12A or extension 12B) and direct shaft 14 to enter one or more body passages of a patient. If desired or necessary, the medical professional may deliver one or more medical tools or devices (e.g., baskets, forceps, etc.) through port 18 and working channel 30 so as to perform a medical procedure. Additionally, if it is determined desirable or necessary, the medical professional may cause one or more of an irrigation fluid, aspiration fluid, or medicament infusion to be delivered through tubes 44 and distally of shaft 14. Once a medical procedure has been completed, the medical professional may retract shaft 14 from the patient, uncouple umbilicus 50 from plug 20, and discard or throw away the entirety of hollow handle 12, including plug 20 and shaft 14. In such a manner, cross-contamination and insufficient sterilization concerns are alleviated.

While principles of the present disclosure are described herein with reference to illustrative examples for particular applications, it should be understood that the disclosure is not limited thereto. Those having ordinary skill in the art and access to the teachings provided herein will recognize additional modifications, applications, embodiments, and substitution of equivalents all fall within the scope of the features described herein. Accordingly, the claimed features are not to be considered as limited by the foregoing description.

We claim:

1. An insertion device, comprising:
a hollow handle;
a shaft extending distally of the hollow handle; and
a cap coupled to a distal end of the shaft, the cap including a camera and an LED coupled thereto.

2. The insertion device of claim 1, wherein the shaft is a rigid shaft.

3. The insertion device of claim 1, wherein the hollow handle includes a rounded ball-head and an extension depending therefrom.

4. The insertion device of claim 3, wherein the shaft extends from a location within the ball-head and through a side of the ball-head of the hollow handle.

5. The insertion device of claim 4, wherein a port extends from a side of the ball-head of the hollow handle opposite the side from which the shaft extends.

6. The insertion device of claim 5, wherein a proximal end of a working channel is coupled to the port and a distal end of the working channel is received within the shaft or cap.

7. The insertion device of claim 3, wherein the hollow handle includes a plug extending from a portion of the extension of the hollow handle opposite the ball-head.

8. The insertion device of claim 7, wherein the plug is selectively coupleable and uncouplable from an umbilicus connector.

9. The insertion device of claim 7, wherein the handle includes two fluid ports proximate the plug.

10. The insertion device of claim 9, wherein each of the two fluid ports is fluidly coupled to a tube extending through the hollow handle, through the shaft, and toward the cap.

11. The insertion device of claim 1, wherein the LED is a first LED, the insertion device further including a second LED disposed on the cap.

12. The insertion device of claim 11, wherein each of the first and second LEDs is disposed on opposing sides of the camera.

13. The insertion device of claim 1, wherein a working channel extends through and proximally of a proximal end of the shaft.

14. An insertion device, consisting essentially of:
a handle, the handle including:
a head having a port extending from a first side of the head; and
a grip depending from the head;
a shaft extending distally of the hollow handle; and
a cap coupled to a distal end of the shaft, the cap including a camera and a light sourced coupled thereto.

15. The insertion device of claim 14, wherein a proximal end of the shaft is disposed within the rounded ball-head of the handle.

16. The insertion device of claim 14, wherein a plug extends from the grip in a direction opposite of the ball-head, wherein the plug is selectively coupleable and uncouplable from an umbilicus connector.

17. The insertion device of claim 16, wherein the grip includes two ports proximate the plug, wherein each of the two ports is fluidly coupled to a tube extending through the handle, through the shaft, and toward the cap.

18. An insertion device, comprising:
a handle, the handle consisting essentially of:
a rounded ball-head having a port extending from a first side of the rounded ball-head; and
a grip depending from the rounded ball-head, the grip including a pair of fluid ports and a plug extending from the grip in a direction opposite that of the rounded ball-head;
a shaft having a proximal end disposed within the rounded ball-head and extending from a second side of the ball-head in a direction opposite the first side of the rounded ball-head; and
a cap coupled to a distal end of the shaft, the cap including a camera and a light source coupled thereto.

19. The insertion device of claim 18, wherein a proximal end of a working channel is coupled to the port and extends distally through a proximal end of the shaft and towards the cap.

20. The insertion device of claim 18, wherein the handle includes a plug extending from a portion of the extension of the handle opposite the ball-head, and wherein a cable or a bundle of cables has a first end coupled to at least one of the camera and light source and a second end coupled to the plug.
